(19) United States
(12) Patent Application Publication
(10) Pub. No.: US 2007/0042453 A1
(43) Pub. Date: Feb. 22, 2007

(54) HIGH THROUGHPUT SCREENING METHOD FOR ANTIMICROBIAL FORMULATIONS

(76) Inventors: John S. Novak, Naperville, IL (US); James T.C. Yuan, Naperville, IL (US)

Correspondence Address: Linda K. Russell, Air Liquide Intellectual Property Dept., 2700 Post Oak Blvd., Suite 1800, Houston, TX, 77056, US

(21) Appl. No.: 11/424,512

(22) Filed: Jun. 15, 2006

Related U.S. Application Data
(60) Provisional application No. 60/710,340, filed on Aug. 22, 2005.

(51) Int. Cl. C12Q 1/18; C12M 1/34

(52) U.S. Cl. 435/32; 435/287.1

(57) ABSTRACT

Methods and systems are provided for increasing the speed and throughput at which the antimicrobial effects of chemical formulations can be evaluated. Generally, the invention provides a method for screening antimicrobial chemical formulations, comprising depositing microorganisms on a slide, depositing chemical formulations onto the deposited microorganisms, removing the chemical formulations from the deposited microorganisms after a given treatment time, depositing fluorescent dyes onto the deposited microorganisms after removing the chemical formulations, and scanning the slide for fluorescence to evaluate viability of the microorganisms after depositing the fluorescent dyes.

### CROSS-REFERENCE TO RELATED APPLICATIONS

This application claims the benefit under 35 U.S.C. § 119(e) to provisional application No. 60/710,340, filed Aug. 22, 2005, the entire contents of which are incorporated herein by reference.

### BACKGROUND

1. Field of the Invention

The invention relates to the improvement of antimicrobial screening techniques for chemical formulations and more specifically, to a method of increasing the speed and throughput at which the antimicrobial effects of chemical formulations can be evaluated.

2. Description of the Related Art

The discovery of new sanitizing agents for industrial and household applications involves antimicrobial screening. Antimicrobial screening provides a way to evaluate the ability of various chemical compounds to inhibit bacterial growth. Normally there is a very slow turnaround for evaluating effects of sanitizers such as ethanol, ozone, chlorine, various salts, acids, bases, and reducing agents on microorganism survival. Traditionally, the antimicrobial effects of chemical compounds are tested individually on pure cultures of select microorganisms of interest by exposure followed by growth and evaluation hours to days later using a selective growth medium.

One method of measuring the antimicrobial effectiveness of a chemical agent is to determine its zone of inhibition. In the agar diffusion method, one species of microorganism is uniformly swabbed onto a nutrient agar plate. A chemical agent is placed on paper disks. These discs are added to the surface of the agar. During incubation, the chemical agent diffuses from the disk into the surrounding agar. An effective agent will inhibit bacterial growth, and measurements can be made to quantify the size of the zones of inhibition around the disks. The relative effectiveness of a compound is determined by comparing the diameter of the zone of inhibition with values in a standard table. Zones of inhibition with larger diameters indicate stronger and more effective antimicrobial agents.

While the agar diffusion method measures relative antimicrobial effectiveness, the dilution method is used to determine whether a chemical is bactericidal (kills bacteria) or bacteriostatic (inhibits bacteria). In the dilution method, the chemical compound of interest is placed in a tube containing the microorganism which is being tested. The microorganism is then deposited onto a nutrient agar plate. If the microorganism grows on the nutrient agar the chemical is bacteriostatic; if not, the microorganism was killed by the chemical, in which case the chemical is then termed bactericidal. Unfortunately, treatments followed by plating on selective media for growth usually take more than a few days to obtain results for just one antimicrobial compound.

Therefore, there remains a need for a high throughput screening method to evaluate the antimicrobial effects of chemical formulations on different microorganisms.

### SUMMARY

Embodiments of the invention generally provide methods and systems for increasing the speed and throughput at which the antimicrobial effects of chemical formulations can be evaluated. In one embodiment, the invention provides a method for screening antimicrobial chemical formulations, comprising depositing microorganisms on a slide, depositing chemical formulations onto the deposited microorganisms, removing the chemical formulations from the deposited microorganisms after a given treatment time, depositing fluorescent dyes onto the deposited microorganisms after removing the chemical formulations, and scanning the slide for fluorescence to evaluate viability of the microorganisms after depositing the fluorescent dyes.

In another aspect, the invention provides a method for screening antimicrobial chemical formulations, comprising placing a slide in an atmosphere controlled chamber, depositing microorganisms on a slide, depositing chemical formulations onto the deposited microorganisms, removing the chemical formulations from the deposited microorganisms after a given treatment time, depositing fluorescent dyes onto the deposited microorganisms after removing the chemical formulations, and scanning the slide for fluorescence to evaluate viability of the microorganisms after depositing the fluorescent dyes.

In another aspect, the invention provides a system for evaluating the antimicrobial effects of numerous chemical formulations, comprising a microarray printer to deposit a plurality of spots containing combinations of chemical formulations, microorganisms, and fluorescent dyes onto a slide, a microarray scanner to detect live and dead microorganisms in each spot through fluorescence markers, and an execution unit which, upon executing code, is configured to analyze the fluorescence marker data of each spot collected from the microarray scanner.

### BRIEF DESCRIPTION OF THE DRAWINGS

For a further understanding of the nature and objects of the present invention, reference should be made to the following detailed description, taken in conjunction with the accompanying drawings, in which like elements are given the same or analogous reference numbers and wherein:

FIG. 1 is a flow diagram showing a system according to one embodiment of this invention.

FIG. 2 exhibits the main processing steps entailed by the embodiments of the invention.

FIG. 3 shows a slide used in one embodiment of the invention, with an enlarged view of a sample spot showing a sanitizer/microorganism mixture.

FIG. 4 shows a slide used in one embodiment of the invention, with live cells and dead cells represented by open circles and solid circles, respectively.

### DESCRIPTION OF PREFERRED EMBODIMENTS

The words and phrases used herein should be given their ordinary and customary meaning in the art by one skilled in the art unless otherwise further defined.

Normally, there is a very slow turnaround for evaluating effects of antimicrobial agents such as ethanol, ozone, chlorine, salts, acids, bases, and reducing agents on microorganism survival. Exposing microorganisms to antimicrobial compounds and plating the treated microorganisms on selective media for growth is a lengthy screening process, and efficacy results for just one antimicrobial compound usually takes more than a few days. Embodiments of the present invention enable rapid, automated microbial evaluation of thousands of chemical formulations at once. A particular embodiment of the invention involves the use of a microarray printer to deposit thousands of sanitizer/microorganism combinations on a slide. A microarray scanner is then used to detect live and dead specimens on the slide as indicated by a viability dye. By scanning thousands of spots in a short period of time, the microarray scanner analysis can expedite the time needed for evaluations of microbial susceptibility to chemical formulations.

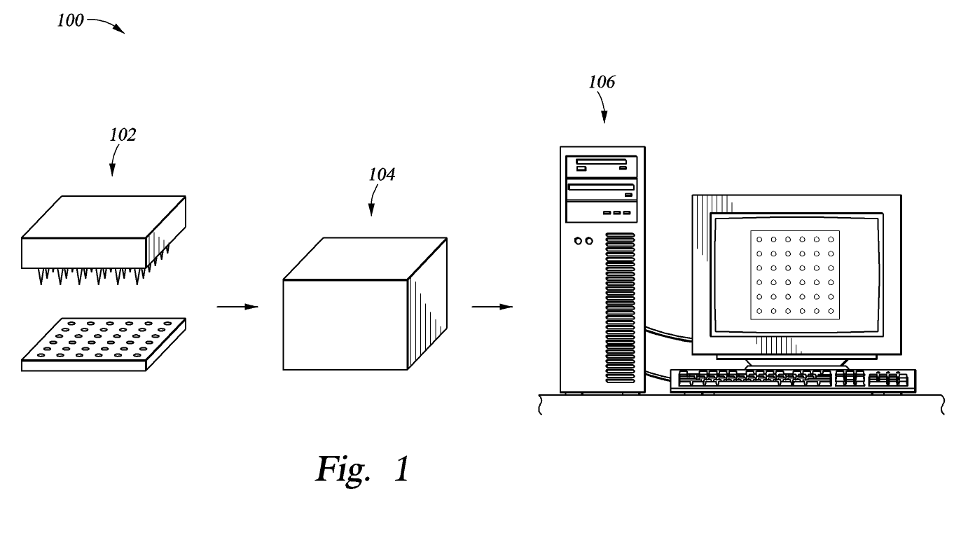

FIG. 1 is a flow diagram showing a system 100, according to one embodiment of this invention. The system 100 includes a microarray printer 102 to deposit thousands of spots containing various combinations of chemical formulations, microorganisms, and fluorescent dyes onto a slide. The system 100 further includes a microarray scanner 104 to detect live and dead microorganisms in each spot through fluorescence markers, and an execution unit 106 which, upon executing a software program, is configured to analyze the fluorescence marker data of each spot collected from the microarray scanner.

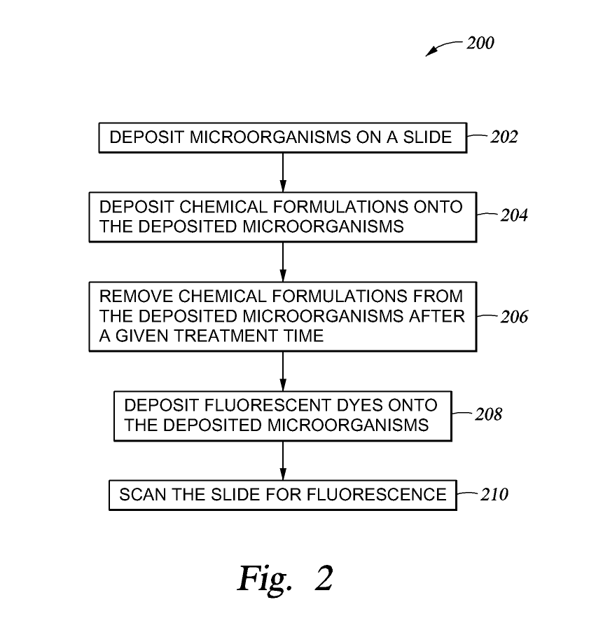

FIG. 2 is a flow diagram of a process 200, according to one embodiment of the present invention. The process 200 includes a processing step 202 involving a deposition of microorganisms on a slide and a processing step 204 involving a deposition of chemical formulations onto the deposited microorganisms. The process 200 further includes a processing step 206 involving a removal of the chemical formulations from the slide after a given treatment time, a processing step 208 involving a deposition of fluorescent dyes onto the deposited microorganisms and an uptake of the dyes by the deposited microorganisms. The process 200 also includes a processing step 210 involving a scanning of the slide for fluorescence to evaluate viability of the microorganisms and efficacy of the antimicrobial formulation.

The processing steps 202-210 according to the embodiments of the invention are described below. The embodiments described herein are provided to illustrate the invention and the particular embodiments shown should not be used to limit the scope of the invention.

The first processing step 202 of the invention involves depositing selected microorganisms on a slide. Microorganisms of concern, such as Escherichia coli, Salmonella, Campylobacter, Listeria monocytogenes, Staphylococcus aureus, and various species of Candida, Bacilli, Clostridia and Enterobacter sakazakii can be deposited on slides to form an array of spots, or a microarray. One embodiment of this invention employs the use of an inkjet printer to deposit solutions containing microorganisms on a slide. Various mixtures of microorganisms can be spotted on slides using an inkjet printer that enables 100 μm spots and up to 10,000 different spots per standard microscope slide (1 mm×25 mm×76 mm). Microorganisms can be deposited at each spot in different concentrations or combinations to approximate realistic environmental conditions. The microorganisms should be dried onto the slide surface prior to depositing any antimicrobial compounds. The specimens can be quick-dried in a dryer void of contamination.

An example of a printer consists of an inkjet head with nozzles through which a solution containing the microorganisms can be deposited on the slide. The nozzles are controlled by a computer which directs where on the slide to deposit the microorganisms, and how much solution to deposit. The printer also contains cartridges that act as reservoirs holding a supply of solution containing the desired microorganisms. After a first set of microorganisms is deposited, the reservoirs are emptied and rinsed. The inkjet head is then washed and flushed thoroughly so that there is no cross-contamination with a next round of microorganisms to be deposited.

The deposition of microorganisms on the slide is preferably conducted in an atmosphere/gas-controlled chamber. It is important to control contamination, humidity, and dust during spotting of the slides. Foreign substances can contaminate the deposited antimicrobial compound/microorganism mixtures. Dust can interfere with data collection from the microarray scanner later in the process. Humidity must be maintained below 75% in order to prevent condensation on the slide. Certain gas mixtures containing variations in nitrogen, hydrogen, carbon dioxide, and oxygen from standard air will be more effective in preparing the slides for analyses. Ideally the deposition process can be performed at room temperature.

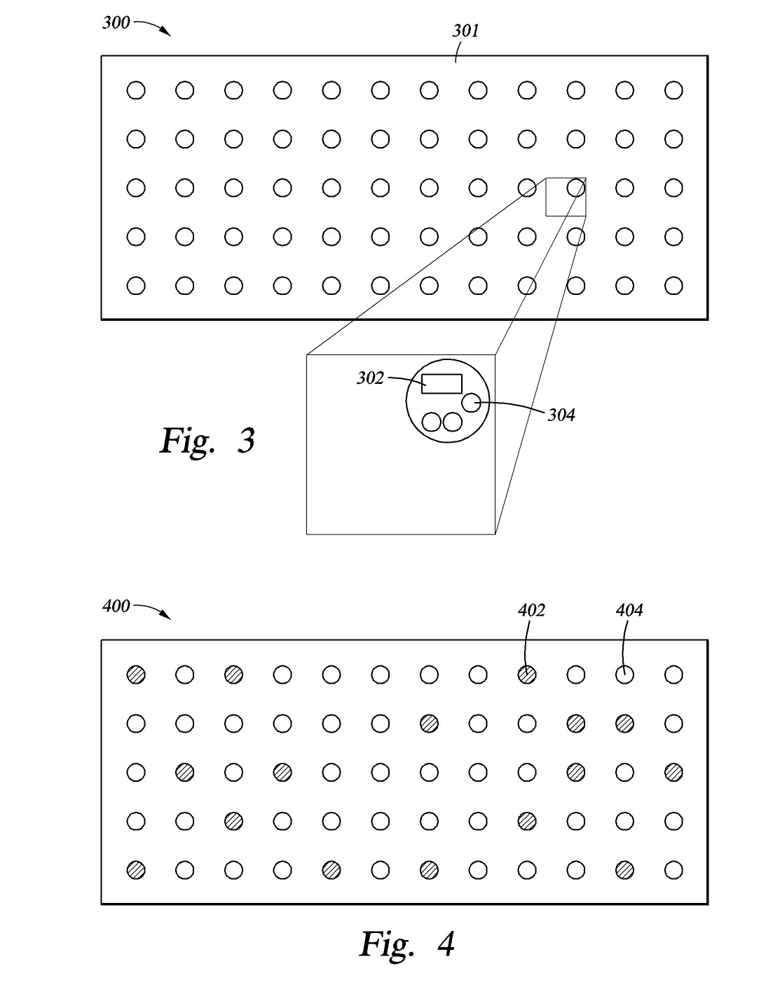

The next main processing step 204 of the invention involves depositing various antimicrobial chemicals on the microorganisms in exact recorded locations on the slide. Examples of antimicrobial formulations that can be deposited include antibiotics, quaternary ammonium compounds, ethanol, reducing agents or other chemical combinations. The chemical permutations may be added directly on top of specific microorganisms dried on the slide. As an example according to one embodiment of this invention, FIG. 3 shows a slide 300 with deposited spots, including a sample spot 301. A sample spot showing a mixture containing an antimicrobial compound 302 and deposited microorganism 304 is enlarged. The antimicrobial compounds can be deposited in exact recorded locations of the slide using the same method to deposit microorganisms in processing step 202. An inkjet printer head can be controlled by a computer (e.g., the execution unit of FIG. 1) which directs where to deposit the antimicrobial compounds suspended in solution, and how much solution to deposit. Following varying treatment exposure times, the slides could be rinsed gently with water to remove the antimicrobial compounds in processing step 206, and quick-dried in a dryer void of contamination to eliminate residual antimicrobial solutions on the slide.

The deposited microorganisms in each sample spot of the microarray are then fluorescently tagged to observe the efficacy of the respective antimicrobial formulation. The processing step 208 of the invention involves depositing fluorescent dyes onto the deposited microorganisms using a microarray printer similar to that used in processing step 202. In one preferred embodiment, Live/Dead BacLight assay reagents (available from Invitrogen Corp., Eugene, Oreg.) can be deposited on each sample spot. The reagents are two fluorescent nucleid acid dyes, Syto 9 and propidium iodide, which indicate cell viability through fluorescence tagging. Syto 9 is a smaller green dye only visible in the absence of propidium iodide, a larger red dye. If cell membranes are damaged, indicating cell death, both fluorescent dyes freely enter the cells to bind nucleic acids, and only red fluorescence is observed. If a cell's membrane is intact, only Syto 9 is small enough to freely enter the cell, and the live undamaged cell would appear to be fluorescent green.

The survival of microorganisms with respect to specific chemical treatments or formulations would then be scanned in processing step 210 using a microarray scanner. In a preferred embodiment, the scanner is tuned to detect Syto 9 green-fluorescent nucleic acid stains at 480-500 nm and propidium iodide red-fluorescent nucleic acid stains at 490-635 nm. FIG. 4 shows a slide 400 as an example according to one embodiment of this invention. The slide 400 is shown after fluorescent tagging, with light spots such as spot 404 representing live microorganisms, and dark spots such as spot 402 representing dead microorganisms. Scanning the slides with a microarray scanner at these wavelengths would result in spots on the slides containing varying numbers of dead (red fluorescence) or live (green fluorescence) cells that could be quantified. Data would be analyzed and stored using customized microarray software programs (such as could be executed on the execution unit of FIG. 1). The resulting data can be used to determine precise microorganism viability relationships with various antimicrobial treatment formulations. In this manner, thousands of treatment permutations can be screened in a matter of hours. The end result is a reduction in costs associated with labor, media, supplies, and time, and an increase in the speed at which new sanitizing agents for industrial or household applications are discovered.

Preferred processes and apparatus for practicing the present invention have been described. It will be understood and readily apparent to the skilled artisan that many changes and modifications may be made to the above-described embodiments without departing from the spirit and the scope of the present invention. The foregoing is illustrative only and that other embodiments of the integrated processes and apparatus may be employed without departing from the true scope of the invention defined in the following claims.

What is claimed is:

1. A method for screening antimicrobial chemical formulations, comprising:
a) depositing microorganisms on a slide;
b) depositing chemical formulations onto the deposited microorganisms;
c) removing the chemical formulations from the deposited microorganisms after a given treatment time;
d) depositing fluorescent dyes onto the deposited microorganisms after removing the chemical formulations; and
e) scanning the slide for fluorescence to evaluate viability of the microorganisms after depositing the fluorescent dyes.

2. The method of claim 1, further comprising drying the slide in a drying chamber in order to stabilize the microorganisms before depositing the chemical formulations.

3. The method of claim 1, wherein the slide is made of glass, metal, plastic, or other polymer material.

4. The method of claim 1, wherein the dimensions of the slide are 1 mm×25 mm×76 mm.

5. The method of claim 1, wherein the microorganisms are deposited using a microarray printer.

6. The method of claim 3, wherein the microarray printer enables the microorganisms to be deposited as spots with a diameter of 100 μm or smaller.

7. The method of claim 3, wherein the microarray printer enables 10,000 or more different spots per slide.

8. The method of claim 1, wherein the microorganisms include one or more of Cryptosporidium parvum oocytes, Escherichia coli, Salmonella, Campylobacter, Listeria monocytogenes, various species of yeast, algae, Protozoa, Streptococcus, Staphylococcus, Pseudomonas, Bacilli, Clostridia, Enterobacter sakazakii, and combinations thereof.

9. The method of claim 1, wherein the microorganisms are deposited on exact recorded locations on the slide.

10. The method of claim 1, wherein the washed slide is dried in a drying chamber.

11. The method of claim 1, wherein the chemical formulations include one or more of sanitizers, quaternary ammonium compounds, ethanol, reducing agents, supercritical CO₂, and combinations thereof.

12. The method of claim 1, wherein removing the chemical formulations from the slide after a given treatment time comprises washing the slides with water.

13. The method of claim 1, wherein the fluorescent dyes are nucleic acid dyes.

14. The method of claim 13, wherein the fluorescent dyes used are Syto 9 and propidium iodide.

15. The method of claim 14, wherein the slide is analyzed for fluorescence of Syto 9 and propidium iodide in a microarray scanner.

16. The method of claim 15, wherein the microarray scanner analyzes fluorescence at wavelengths between about 480 and about 500 nm for Syto 9, and wavelengths between about 490 and about 635 nm for propidium iodide.

17. A method for screening antimicrobial chemical formulations, comprising:
a) placing a slide in an atmosphere controlled chamber;
b) depositing microorganisms on a slide;
c) depositing chemical formulations onto the deposited microorganisms;
d) removing the chemical formulations from the deposited microorganisms after a given treatment time;
e) depositing fluorescent dyes onto the deposited microorganisms after removing the chemical formulations; and
f) scanning the slide for fluorescence to evaluate viability of the microorganisms after depositing the fluorescent dyes.

18. The method of claim 17, further comprising drying the slide in a drying chamber in order to stabilize the microorganisms before depositing the chemical formulations.

19. The method of claim 17, wherein the atmosphere controlled chamber maintains humidity below 75%.

20. The method of claim 17, wherein the atmosphere controlled chamber allows variations in hydrogen, carbon dioxide and oxygen gas content.

21. The method of claim 17, wherein the slide is made of glass, metal, plastic, or other polymer material.

22. The method of claim 17, wherein the dimensions of the slide are 1 mm×25 mm×76 mm.

23. The method of claim 17, wherein the microorganisms are deposited using a microarray printer.

24. The method of claim 17, wherein the microarray printer enables the microorganisms to be deposited as spots with a diameter of 100 μm or smaller.

25. The method of claim 17, wherein the microarray printer enables 10,000 or more different spots per slide.

26. The method of claim 1, wherein the microorganisms include one or more of Cryptosporidium parvum oocytes, Escherichia coli, Salmonella, Campylobacter, Listeria monocytogenes, various species of yeast, algae, Protozoa, Streptococcus, Staphylococcus, Pseudomonas, Bacilli, Clostridia, Enterobacter sakazakii, and combinations thereof.

27. The method of claim 17, wherein the microorganisms are deposited on exact recorded locations on the slide.

28. The method of claim 17, wherein the washed slide is dried in a drying chamber.

29. The method of claim 17, wherein the chemical formulations include one or more of sanitizers, quaternary ammonium compounds, ethanol, reducing agents, supercritical CO₂, and combinations thereof.

30. The method of claim 17, wherein removing the chemical formulations from the slide after a given treatment time comprises washing the slides with water.

31. The method of claim 17, wherein the fluorescent dyes are nucleic acid dyes.

32. The method of claim 30, wherein the fluorescent dyes used are Syto 9 and propidium iodide.

33. The method of claim 31, wherein the slide is analyzed for fluorescence of Syto 9 and propidium iodide in a microarray scanner.

34. The method of claim 32, wherein the microarray scanner analyzes fluorescence at wavelengths between about 480 and about 500 nm for Syto 9, and wavelengths between about 490 and about 635 nm for propidium iodide.

35. A system for evaluating the antimicrobial effects of numerous chemical formulations, comprising:
a) a microarray printer to deposit a plurality of spots containing combinations of chemical formulations, microorganisms, and fluorescent dyes onto a slide;
b) a microarray scanner to detect live and dead microorganisms in each spot through fluorescence markers; and
c) an execution unit which, upon executing code, is configured to analyze the fluorescence marker data of each spot collected from the microarray scanner.
